(19) United States
(12) Patent Application Publication
(10) Pub. No.: US 2004/0191273 A1
(43) Pub. Date: Sep. 30, 2004

(54) Methods and compositions for obtaining disease protection for economically important animals

(76) Inventor: Joseph M. Jilka, College Station, TX (US)

Correspondence Address: MCKEE, VOORHEES & SEASE, P.L.C., 801 GRAND AVENUE, SUITE 3200, DES MOINES, IA, 50309-2721, US

(21) Appl. No.: 10/823,150

(22) Filed: Apr. 13, 2004

Related U.S. Application Data
(63) Continuation of application No. 10/169,813, filed on Jul. 9, 2002.
Application No. 10/169,813 is a 371 of international application No. PCT/US01/01148, filed on Jan. 12, 2001.
(60) Provisional application No. 60/176,220, filed on Jan. 14, 2000.
(60) Provisional application No. 60/196,809, filed on Apr. 13, 2000.

(30) Foreign Application Priority Data
Jan. 12, 2001 (US) US01/01148

(51) Int. Cl. A61K 39/12

(52) U.S. Cl. 424/204.1

(57) ABSTRACT

According to the invention, the oral administration of immunogenic compositions to induce protection against challenge by bacterial, viral or other pathogens, in the absence of an antibody response. In one embodiment the antigenic composition is a transgenic plant expressing an antigenic determinate of said pathogen, in yet another embodiment the antigenic composition is a enterotoxin which may be administered with food or as part of a transgenic plant. According to the invention, administration of these immunogenic compositions in traditionally subclinical amounts which are insufficient to generate an antibody response, provides protection against later challenge by a particular pathogen.

### CROSS-REFERENCE TO RELATED APPLICATION

This application is a Continuation of co-pending, commonly owned U.S. Ser. No. 10/169,813 filed July 9, 2002 which is a U.S. National Phase Application of PCT/US01/01148 filed Jan. 12, 2001 which relies on the priority of Provisional Application Serial Number 60/176,220 filed Jan. 14, 2000, and Provisional Application Serial No. 60/196,809 filed Apr. 13, 2000, the contents of which are hereby incorporated by reference in their entirety.

### FIELD OF THE INVENTION

This invention relates generally to methods for providing disease protection and recovery in animals and humans and more particularly to the administration of oral immunogenic compositions including edible transgenic plants, and bacterial toxins for protection against disease challenge. BACKGROUND OF THE INVENTION

Diseases have been a plague on civilization for thousands of years, affecting not only man but animals. In economically advanced countries of the world, diseases are 1) temporarily disabling; 2) permanently disabling or crippling; or 3) fatal. In the lesser developed countries, diseases tend to fall into the latter two categories, permanently disabling or crippling and fatal, due to many factors, including a lack of preventative immunization and curative medicine.

Vaccines have been administered to humans and animals traditionally to induce their immune systems to produce antibodies against viruses, bacteria, and other types of pathogenic organisms. In the economically advanced countries of the world, vaccines have brought many diseases under control. In particular, many viral diseases are now prevented due to the development of immunization programs. The virtual disappearance of smallpox, certainly, is an example of the effectiveness of a vaccine worldwide. But many vaccines for such diseases as poliomyelitis, measles, mumps, rabies, foot and mouth, and hepatitis B are still too expensive for the lesser developed countries to provide to their large human and animal populations. Lack of these preventative measures for animal populations can worsen the human condition by creating food shortages.

The lesser developed countries do not have the monetary funds to immunize their populations with currently available vaccines. There is not only the cost of producing the vaccine but the further cost of the professional administration of the vaccine. Also, some vaccines require multiple doses to maintain immunity. Therefore, often, the countries that need the vaccines the most can afford them the least.

Underlying the development of any vaccine is the ability to grow the disease causing agent in large quantities. At the present, vaccines are usually produced from killed or live attenuated pathogens. If the pathogen is a virus, large amounts of the virus must be grown in an animal host or cultured animal cells. If a live attenuated virus is utilized, it must be clearly proven to lack virulence while retaining the ability to establish infection and induce humoral and cellular immunity. If a killed virus is utilized, the vaccine must demonstrate the capacity of surviving antigens to induce immunization. Additionally, surface antigens, the major viral particles which induce immunity, may be isolated and administered to proffer immunity in lieu of utilizing live attenuated or killed viruses.

Vaccine manufacturers often employ complex technology entailing high costs for both the development and production of the vaccine. Concentration and purification of the vaccine is required, whether it is made from the whole bacteria, virus, other pathogenic organism or a sub-unit thereof. The high cost of purifying a vaccine in accordance with Food and Drug Administration (FDA) regulations makes oral vaccines prohibitively expensive to produce because they require ten to fifty times more than the regular quantity of vaccine per dose than a vaccine which is parenterally administered. Of all the viral vaccines being produced today only a few are being produced as oral vaccines.

According to FDA guidelines, efficacy of vaccines for humans must be demonstrated in animals by antibody development and by resistance to infection and disease upon challenge with the pathogen. When the safety and immunogenicity levels are satisfactory, FDA clinical studies are then conducted in humans. A small carefully controlled group of volunteers are enlisted from the general population to begin human trials. This begins the long and expensive process of testing which takes years before it can be determined whether the vaccine can be given to the general population. If the trials are successful, the vaccine may then be mass produced and sold to the public.

Even after these precautions are taken, problems can arise. With the killed virus vaccines, there is always a chance that one of the live viruses has survived and vaccination may lead to isolated cases of the disease. Moreover, since both the killed and live attenuated types of virus vaccines are made from viruses grown in animal host cells, the vaccines are sometimes contaminated with cellular material from the animal host which can cause adverse, sometimes fatal, reactions in the vaccine recipient. Legal liability of the vaccine manufacturer for those who are harmed by a rare adverse reaction to a new or improved vaccine necessitates expensive insurance which ultimately adds to the cost of the vaccine.

Some vaccines have other disadvantages. Vaccines prepared from whole killed virus generally stimulate the development of circulating antibodies (IgM, IgG) thereby conferring a limited degree of immunity which usually requires boosting through the administration of additional doses of vaccine at specific time intervals. Live attenuated viral vaccines, while much more effective, have limited shelf-life and storage problems requiring maintaining vaccine refrigeration during delivery to the field.

Efforts today are being made to produce less expensive vaccines which can be administered in a less costly manner. Recombinants or mutants can be produced that serve in place of live virus vaccines. The development of specific deletion mutants that alter the virus, but do not inactivate it, yield vaccines that can replicate but cannot revert to virulence.

Recombinant DNA techniques are being developed to insert the gene coding for the immunizing protein of one virus into the genome of a second, avirulent virus type that can be administered as the vaccine. Recombinant vaccines may be prepared by means of a vector virus such as vaccinia virus or by other methods of gene splicing. Vectors may include not only avirulent viruses but bacteria as well. A live recombinant hepatitis A vaccine has been constructed using attenuated Salmonella typhimurium as the delivery vector via oral administration.

Various avirulent viruses have been used as vectors. The gene for hepatitis B surface antigen (HBsAg) has been introduced into a gene non-essential for vaccinia replication. The resulting recombinant virus has elicited an immune response to the hepatitis B virus in test animals. Additionally, researchers have used attenuated bacterial cells for expressing hepatitis B antigen for oral immunization. Importantly, when whole cell attenuated Salmonella expressing recombinant hepatitis antigen were fed to mice, anti-viral T and B cell immune responses were observed. These responses were generated after a single oral immunization with the bacterial cells resulting in high-titers of the antibody. See, e.g., “Expression of hepatitis B virus antigens in attenuated Salmonella for oral immunization,” F. Schodel and H. Will, Res. Microbiol., 141:831-837 (1990). Others have had similar success with oral administration routes for recombinant hepatitis antigens. See, e.g., M. D. Lubeck et al., “Immunogenicity and efficiacy testing in chimpanzees of an oral hepatitis B vaccine based on live recombinant adenovirus,” Proc. Natl. Acad Sci. 86:6763-6767 (1989); S. Kuriyama, et al., “Enhancing effects of oral adjuvants on anti-HBs responses induced by hepatitis B vaccine,” Clin. Exp. Immunol. 72:383-389 (1988).

Other virus vectors may possess large genomes, e.g. the herpesvirus. The oral adenovirus vaccine has been modified so that it carries the HBsAg immunizing gene of the hepatitis B virus. Chimeric polio virus vaccines have been constructed of which the completely avirulent type 1 virus acts as a vector for the gene carrying the immunizing VP1 gene of type 3.

Recent advances in genetic engineering have provided the requisite tools to transform plants to contain foreign genes. Plants that contain the transgene in all cells can then be regenerated and can transfer the transgene to their offspring in a Mendelian fashion. Both monocotyledenous and dicotyledenous plants have been stably transformed. Some examples to date are the production of interferon in tobacco (Goodman et al., 1987), enkephalins in tobacco, Brassica napus and Ababidopsis thaliana (Vandekerchove et al., 1989), human serum albumin in tobacco and potato (Sijmons et al., 1990) antibodies in tobacco (Hiatt et al., 1990) and hepatitis B antigen (Mason et al., 1992). The use of transgenic plants for producing vaccines has been suggested; however, there has been no showing in these references of expression in plants at levels sufficient to protect animals against disease or that oral immunization with the plant would be effective to protect animals, particularly domestic animals, against disease.

Attempts to produce transgenic plants expressing bacterial antigens of Escherichia coli and of Streptococcus mutans have been made (Curtiss and Ihnen, WO 90/0248, 22 Mar. 1990). Additionally viral pathogens have also been successfully introduced into plants. See U.S. Pat. Nos. 5,612,487; 5,484,719, 5,914,123; 6,034,298; 6,136,320; and PCT published application PCT/US96/14662.

Thus, there is a continuing need in the art for obtaining less expensive and more accessible vaccines with improved safety. Prior efforts for production of vaccines have routinely focussed on serum antibodies to pronouce the effectiveness of the agent for protection against infection. This has often required the use of adjuvants and adminstiration (often intraveously or subcutaneously) of large amounts of antigen to ensure that a serum specific antibody response is generated. Applicants have identified that infact, protection similar to that with traditional vaccines can be achieved in the absense of an antibody repsonse. In fact, the need for specificity of antigen is not always necessary and protection can be achieved with administration of bacterial toxins or adjuvants themselves.

It is an object of the present invention to provide orally administered compositions for protection against disease caused by pathogens.

It is yet another object of the invention to provide improved safety by oral administration of sub-critical doses of transgenic plants which express antigens for protection against pathogenic disease.

It is yet another object of the invention to provide such protection in the absence of an antibody response.

It is yet another object of the invention to provide methods and compositions for providing protection against interferon-sensitive pathogens by administration of a bacterial toxin.

These and other objects of the invention will become apparent from the detailed description of the invention which follows:

### SUMMARY OF THE INVENTION

According to the invention, the oral administration of immunogenic compositions to induce protection against challenge by bacterial, viral or other pathogens, in the absence of an antibody response is presented. In one embodiment the antigenic composition is an edible plant expressing an antigenic determinant of said pathogen, in yet another embodiment the immunogenic composition is a enterotoxin which may be administered with food or as part of a transgenic plant. According to the invention, administration of these immunogenic compositions in amounts insufficient to generate, and surprisingly in the absense of, an antibody response, provides protection against later challenge by a particular pathogen. In the case of enterotoxin, the response is not necessarily antigen specific as a bacterial enterotoxin was found sufficient to provide protection against a later viral challenge. Viral pathogens subject to this nonspecific response to enterotoxin include any of the group of viral agents which are susceptible to increased interferon levels including but not limited to: transmissible gastroenteritis virus (TGEV), porcine reproductive and respiratory syndrome, (PRRS), swine arterivirus, and the class of corona viruses and rotaviruses. Other such virsuses are described in Derbyshire J. B. 1989 “The interferon selectivity of sensitivity of selected procine viruses” Can. J. Vet. Res. 53:52-55.

For purposes of this application the following terms shall have the definitions recited herein. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. The terms defined below are more fully defined by reference to the specification as a whole.

As used herein the term “bacterial enterotoxin” shall include any of the family of bacterial protein toxins composed of one A polypeptide and five B polypeptides maintained by noncovalent bonding in a quaternary structure between the A subunit and a pentameric ring of B subunits. This includes but is not limited to the heat labile enterotoxins from Escherichia coli. Further information about these toxins is disclosed in the following which are hereby incorporated in their entirety by reference: Spanger, B. D. 1992 “Structure and function of cholera toxin and the related Eschericia coli heat-labile enterotoxins”. Microbiol. Rev. 56:622-647; Gill, D. M., J. D. Clements, D. C. Robertson, and R. A. Finkelstein. 1981. “Subunit number and arrangements in Escherichia coli heat-labile enterotoxin”. Infect. Immun. 56:1748-1753; Hardy, S. J., J. Holmgren, S. Johansson, J. Sanchez and T. R. Hirst. 1988. “Coordinated assembly of multisubunit proteins: oligomerization of bacterial enterotoxins in vivo and in vitro”. Proc. Natl. Acad. Sci. USA 85: 7109-7113; and Sixma, T. K., S. E. Pronk, K. H. Kalk, E. S. Wartna, B. A. van Zanten,B. Witholt, and W. G. Hol. 1991. “Crystal structure of a cholera toxin-related heat-labile enterotoxin from E. coli”. Nature 351:371-377. This term is also intended to include recombinant as well as conservatively modified variants and other peptide variants which retain the interferon stimulating activity of the protein. The amino acid and nucleotide sequences encoding these enzymes are generally know to those of skill in the art and available through sources such as Genbank and the references disclosed herein. Those of skill in the art will appreciate that enterotoxins will be applicable to the teachings herein, or will become available or isolated using no more than routine experimentation.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1.993). The product of amplification is termed an amplicon.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). Thus, the maize preferred codon for a particular amino acid may be derived from known gene sequences from maize. Maize codon usage for 28 genes from maize plants are listed in Table 4 of Murray et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells or insect cells.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “nucleic acid” or “nucleotide” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” can include reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. Particularly preferred plants are agricultural plants.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

A “structural gene” is a DNA sequence that is transcribed into messenger RNA (mRNA) which is then translated into a sequence of amino acids characteristic of a specific polypeptide.

The term “expression” refers to biosynthesis of a gene product. Structural gene expression involves transcription of the structural gene into mRNA and then translation of the mRNA into one or more polypeptides.

A “cloning vector” is a DNA molecule such as a plasmid, cosmid, or bacterial phage that has the capability of replicating autonomously in a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance or ampicillin resistance.

An “expression vector” is a DNA molecule comprising a gene that is expressed in a host cell. Typically, gene expression is placed under the control of certain regulatory elements including promoters, tissue specific regulatory elements, and enhancers. Such a gene is said to be “operably linked to” the regulatory elements.

A “recombinant host” may be any prokaryotic or eukaryotic cell that contains either a cloning vector or an expression vector. This term also includes those prokaryotic or eukaryotic cells that have been genetically engineered to contain the cloned genes in the chromosome or genome of the host cell.

As used herein the term “protection” refers to an improvement in general health and vigor of an animal after challenge with a pathogen as compared to the health and vigor of a nonprotected animal after challenge evidenced by things like weight gain, a decrease in clinical symptoms, lack of virus shedding, and/or other such observations known to those of skill in the art as criteria for establishing disease protection after challenge.

### DETAILED DESCRIPTION OF THE FIGURES

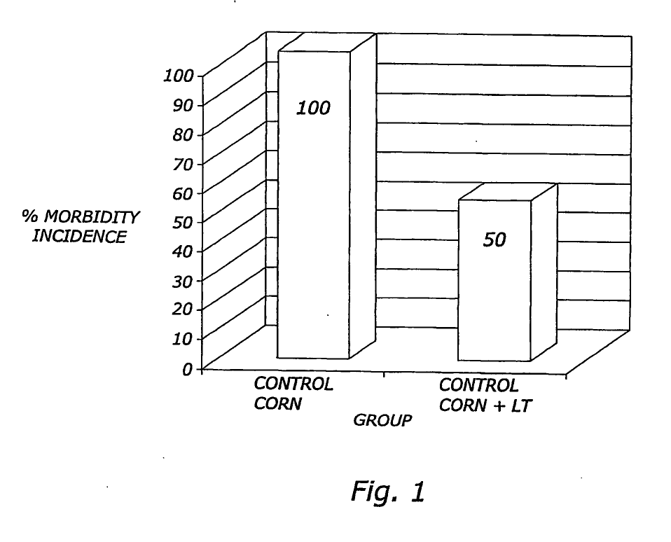

FIG. 1 is a chart showing the percent of morbidity incidence in each of the treatment groups. This data is compilation of the significant (>2) clinical scores for each treatment group. This data shows that 100% of the pigs that were fed control corn developed TGEV clinical symptoms. Only 50% of those that received the Lt exhibited symptoms.

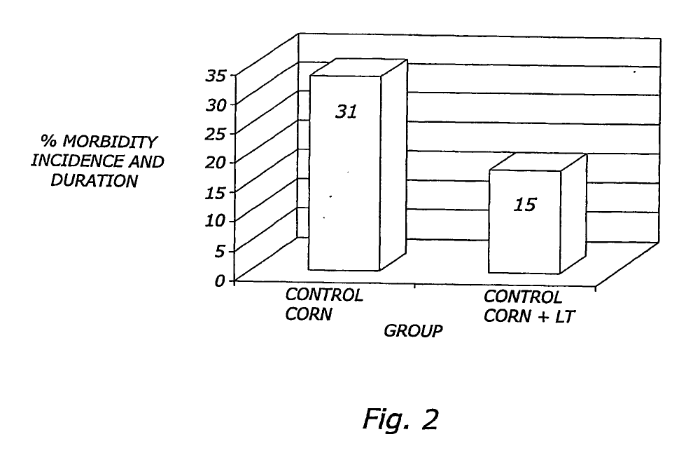

FIG. 2 is a chart showing the percent of morbidity incidence and duration in each of the treatment groups. This data is compilation of the significant (>2) clinical scores for each treatment group. This data shows that pigs that were fed control corn developed TGEV clinical symptoms while those that received the control Lt exhibited fewer symptoms.

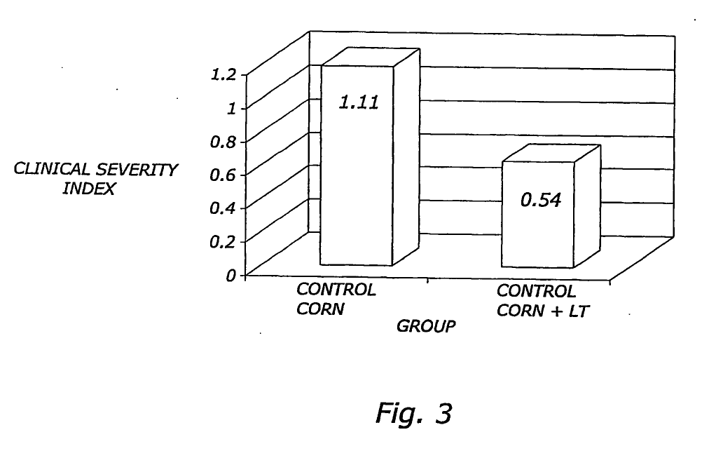

FIG. 3 is a chart showing the clinical severity index for each treatment group. This data is compilation of the total clinical value divided by the total number of pig days for each treatment group. This data shows that the disease that developed in the pigs that were fed control corn was much higher than the Lt group.

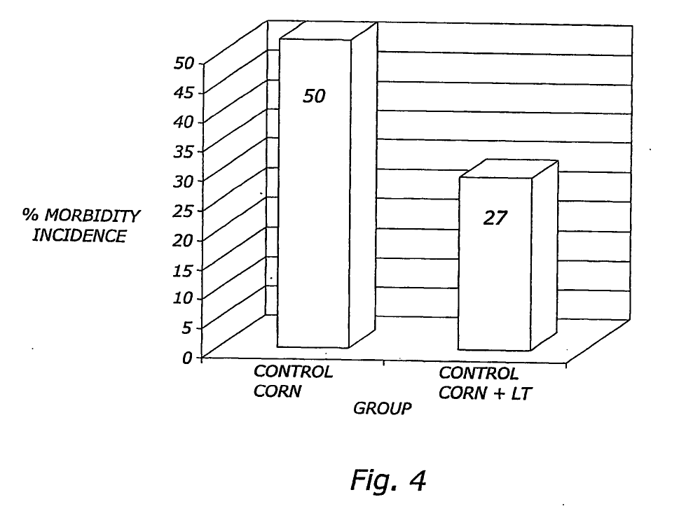

FIG. 4 is a chart showing the percent of morbidity incidence in each of the treatment groups. This data is compilation of the significant (>2) clinical scores for each treatment group. This data shows that 50% of the pigs that were fed control corn developed TGEV clinical symptoms. Only 27% of those that received the Lt exhibited symptoms.

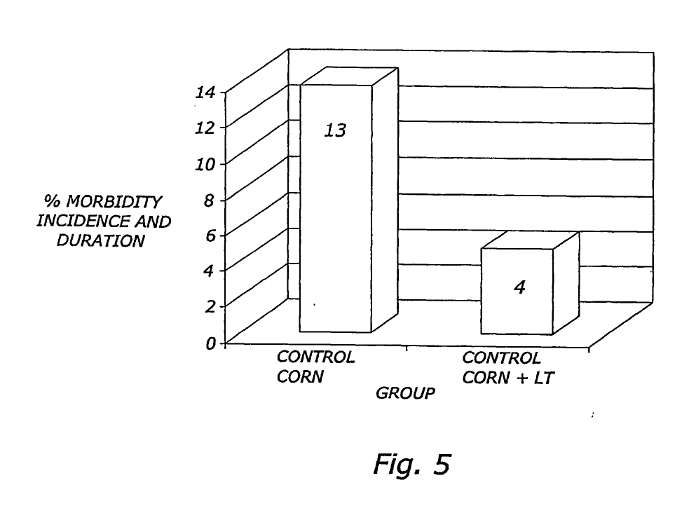

FIG. 5 is a chart showing the percent of morbidity incidence and duration in each of the treatment groups. This data is compilation of the significant (>2) clinical scores for each treatment group. This data shows that pigs that were fed control corn developed TGEV clinical symptoms while those that received the control Lt exhibited fewer symptoms.

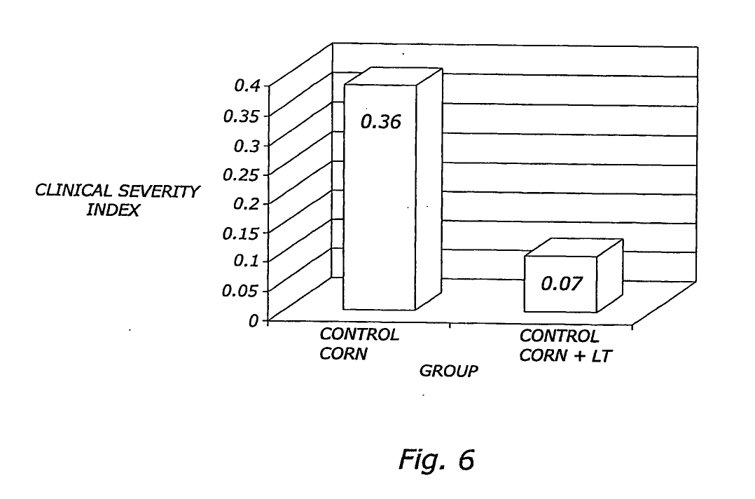

FIG. 6 is a chart showing the clinical severity index for each treatment group. This data is compilation of the total clinical value divided by the total number of pig days for each treatment group. This data shows that the disease that developed in the pigs that were fed control corn was much higher than the Lt group.

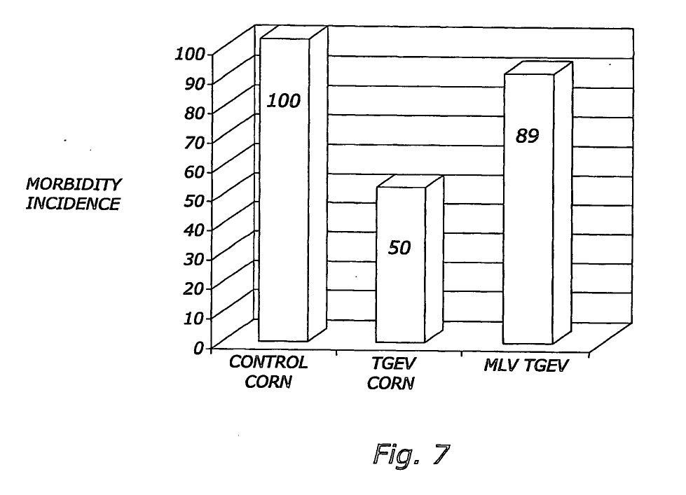

FIG. 7 is a morbidity incidence chart showing the percent of morbidity incidence in each of the treatment groups. This data is compilation of the significant (>2) clinical scores for each treatment group. This data shows that 100% of the pigs that were fed control corn developed TGEV clinical symptoms. Only 50% of those that received the TGEV corn exhibited symptoms and 89% of the pigs receiving the modified live vaccine developed symptoms.

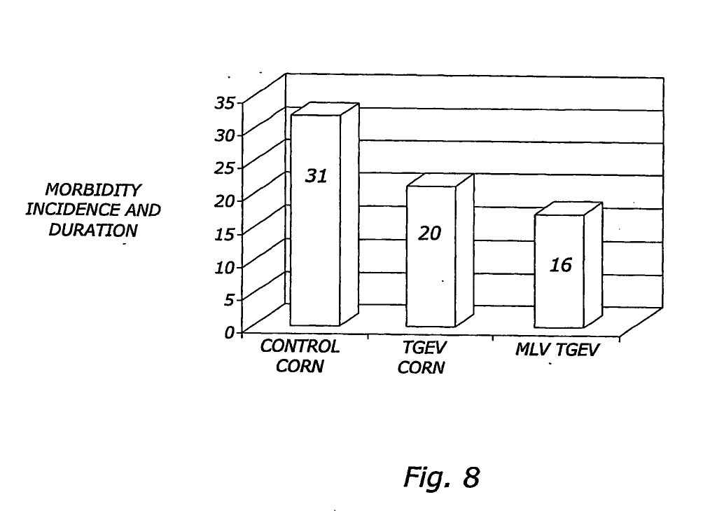

FIG. 8 is a mobidity incidence and duration chart showing the percent of morbidity incidence and duration in each of the treatment groups. This data is compilation of the significant (>2) clinical scores for each treatment group.

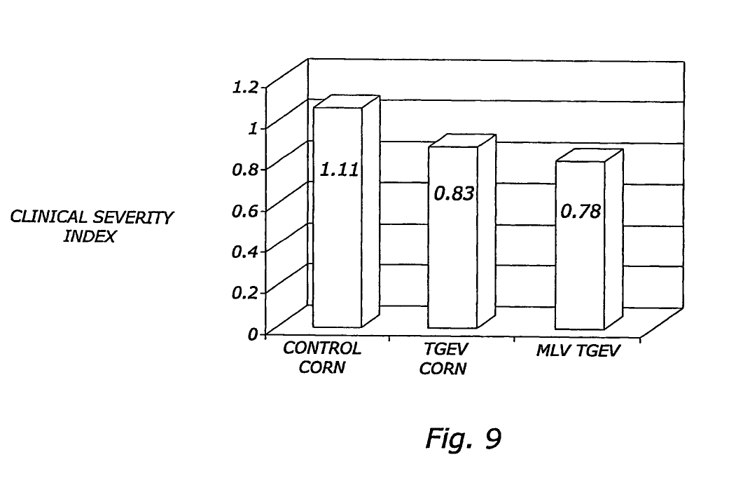

FIG. 9 is a clinical severity index chart showing the clinical severity index for each treatment group. This data is compilation of the total clinical value divided by the total number of pig days for each treatment group. This data shows that the disease that developed in the pigs that were fed control corn was much higher than either the TGEV-S containing transgenic corn or the modified live vaccine treatment group.

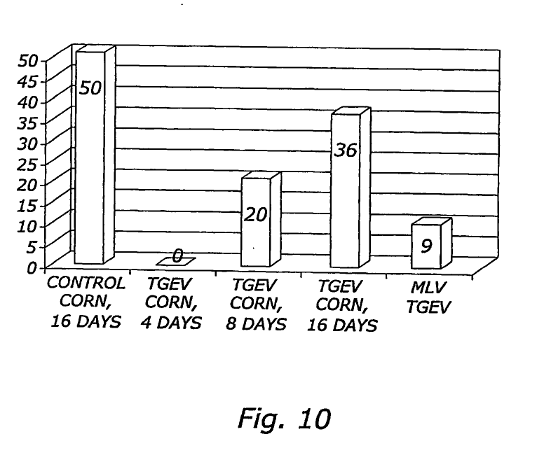

FIG. 10 is a morbidity incidence chart showing the percent of morbidity incidence in each of the treatment groups. This data is compilation of the significant (>2) clinical scores for each treatment group. This data shows that 50% of the pigs that were fed control corn developed TGEV clinical symptoms. 0%, 20% and 36% of the pigs that received 4 days, 8 days and 16 days of TGEV corn, respectively, while 9% of the pigs receiving the modified live vaccine developed symptoms.

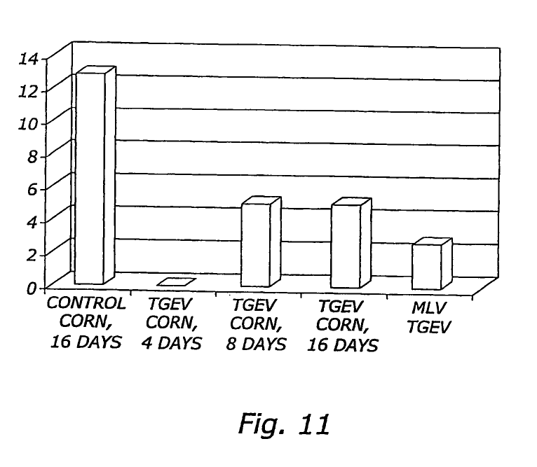

FIG. 11 is a morbidity incidence and duration chart showing the percent of morbidity incidence and duration in each of the treatment groups. This data is compilation of the significant (>2) clinical scores for each treatment group.

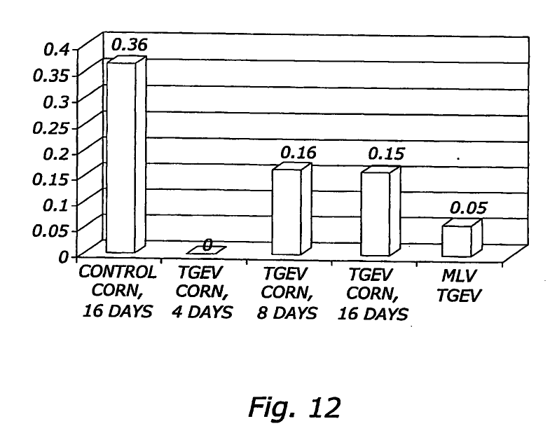

FIG. 12 is a clinical severity index chart showing the clinical severity index for each treatment group. This data is compilation of the total clinical value divided by the total number of pig days for each treatment group. This data shows that the disease that developed in the pigs that were fed control corn was much higher than either the TGEV-S containing transgenic corn or the modified live vaccine treatment group.

### DETAILED DESCRIPTION OF THE INVENTION

The present invention has several components which include: using recombinant DNA techniques to create a plasmid vector which contains a DNA segment encoding one or more antigenic proteins which confer immunity in a human or an animal to a particular disease and for the expression of antigenic protein(s) in desired tissues of a plant; selecting an appropriate host plant to receive the DNA segment encoding antigenic protein(s) and subsequently produce the antigenic protein(s); transferring the DNA segment encoding the antigenic protein(s) from the plasmid vector into the selected host plant; regenerating the transgenic plant thereby producing plants expressing the antigenic protein(s) which functions as a vaccine(s); and administering an edible part of the transgenic plant containing the antigenic protein(s) as an oral immunogenic composition to either a human or an animal by the consumption of a transgenic plant part. The present invention thereby provides for the production of a transgenic plant which when consumed as food, at least in part, by a human or an animal causes protection against challing with a particular pathogen. This response is characterized by protection without antibodies to the particular disease. The response is the result of the production in the transgenic plant of antigenic protein(s). The production of the antigenic protein(s) is the result of stable genetic integration into the transgenic plant of DNA regions designed to cause regulated expression of antigenic protein(s) in the transgenic plants.

Immunogenic Compositions and Their Administration

The present invention may be used to produce any type immunogenic composition effective in protecting humans and animals against diseases. Viruses, bacteria, fungi, and parasites that cause disease in humans and animals can contain antigenic protein(s) which can confer immunity in a human or an animal to the causative pathogen. A DNA sequence encoding any of these viral, bacterial, fungal or parasitic antigenic proteins may be used in the present invention, but surprisingly, the antigen need not be introduced in a manner to stimulate a specific antibody response.

Mutant and variant forms of the DNA sequences encoding a antigenic protein which confers immunity to a particular virus, bacteria, fungus or parasite in an animal (including humans) may also be utilized in this invention. For example, expression vectors may contain DNA coding sequences which are altered so as to change one or more amino acid residues in the antigenic protein expressed in the plant, thereby altering the antigenicity of the expressed protein. Expression vectors containing a DNA sequence encoding only a portion of an antigenic protein as either a smaller peptide or as a component of a new chimeric fusion protein are also included in this invention.

The present invention is advantageously used to produce viral immunogenic composition for humans and animals. The following table sets forth a list of vaccines now used for the prevention of viral diseases in humans. According to the invention, these can be treated with subclinical doses to achieve protection without sero-conversion.

| | | Condition of | Route of |
|---|---|---|---|
| Disease | Source of Vaccine | Virus | Administration |
| Poliomyelitis | Tissue culture (human diploid cell line, | Live | Oral |
| | monkey kidney) | attenuated | Subcutaneous |
| | | Killed | |
| Measles | Tissue culture (chick embryo) | Live | Subcutaneous |
| | | attenuated | |
| Mumps | Tissue culture (chick embryo) | Live | Subcutaneous |
| | | attenuated | |
| Rubella | Tissue culture (duck embryo, rabbit, or | Live | Subcutaneous |
| | human diploid) | attenuated | |
| Smallpox | Lymph from calf or sheep | Live vaccinia | Intradermal |
| Yellow Fever | Tissue cultures and eggs | Live | Subcutaneous |
| | | attenuated | |
| Viral | Purified HBsAg from “health” carriers | Live | Subcutaneous |
| hepatitis B | Recombinant HBsAg from yeast | attenuated | Subcutaneous |
| | | Subunit | |
| Influenza | Highly purified or subviral forms (chick | Killed | Subcutaneous |
| | embryo) | | |
| Rabies | Human diploid cell cultures | Killed | Subcutaneous |
| Adenoviral | Human diploid cell cultures | Live | Oral |
| infections | | attenuated | |
| Japanese B | Tissue culture (hamster kidney) | Killed | Subcutaneous |
| encephalitis | | | |
| Varicella | Human diploid cell cultures | Live | Subcutaneous |
| | | attenuated | |

The present invention is also advantageously used to produce immunogenic compositions to protect animals. Diseases such as: canine distemper, rabies, canine hepatitis, parvovirus, and feline leukemia may be controlled with proper immunization of pets. Viral vaccines for diseases such as: Newcastle, Rinderpest, hog cholera, blue tongue and foot-mouth can control disease outbreaks in production animal populations, thereby avoiding large economic losses from disease deaths. Prevention of bacterial diseases in production animals such as: brucellosis, fowl cholera, anthrax and black leg through the use of vaccines has existed for many years. Today new recombinant DNA vaccines, e.g. rabies and foot and mouth, have been successfully produced in bacteria and yeast cells and can facilitate the production of a purified vaccine containing only the immunizing antigen. Veterinary vaccines utilizing cloned antigens for protozoans and helminths promise relief from parasitic infections which cripple and kill.

The oral immunogenic composition produced by the present invention is administered by the consumption of the foodstuff which has been produced from the transgenic plant producing the antigenic protein as the vaccine. The edible part of the plant is used as a dietary component while the vaccine is administered in the process. In a second embodiment the enterotoxin may be combined with food stuff including even water for ingestion.

The present invention allows for the production of not only a single vaccine in an edible plant but for a plurality of vaccines into one foodstuff. DNA sequences of multiple antigenic proteins can be included in the expression vector used for plant transformation, thereby causing the expression of multiple antigenic amino acid sequences in one transgenic plant. Alternatively, a plant may be sequentially or simultaneously transformed with a series of expression vectors, each of which contains DNA segments encoding one or more antigenic proteins. For example, there are five or six different types of influenza, each requiring a different vaccine. A transgenic plant expressing multiple antigenic protein sequences can simultaneously elicit an immune response to more than one of these strains, thereby giving disease immunity even though the most prevalent strain is not known in advance.

Immunogenic compositions produced in accordance with the present invention may also be incorporated into the feed of animals. This represents an important means to produce lower cost disease prevention for pets, production animals, and wild species.

Contrary to traditional vaccines, the immune compositions of the present invention will be preferably utilized directly as oral ingestion of transgenic plant material, or immunogenic compositions derived from bacterial enterotoxins. Preparation of the enterotoxin employs purification of the same and may take many forms known well to those of skill in the art, and most are commercially available for use as adjuvants.

The preparation of immunogenic compositions such as vaccines is generally well understood in the art (e.g., those derived from fermentative yeast cells known well in the art of vaccine manufacture cite to Valenzuela et al Nature 298, 347-350 (1982), as exemplified by U.S. Pat. Nos. 4,608,251; 4,601,903; 4,599,231; 4,599,230; 4,596,792; and 4.578,770, all incorporated herein by reference.

Oral formulations other than edible plant portions described in detail herein include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain 10-95% of active ingredient, preferably 25-70%, which may be added to the enterotoxin or plant material.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the immune response.

The course of the immunization and administration ammounts may be determined by assays for antibodies for the supernatant antigens. The assays may be performed by labeling with conventional labels, such as radionuclides, enzymes, fluorescers, and the like. These techniques are well known and may be found in a wide variety of patents, such as U.S. Pat. Nos. 3,791,932; 4,174,384 and 3,949,064, as illustrative of these types of assays. According to the invention, administration is designed to be below levels at which antibodies are generated.

Host Plant Selection

A variety of plant species have been genetically transformed with foreign DNA, using several different gene insertive techniques. Since important progress is being made to clone DNA coding regions for vaccine antigens for parasitic tropical diseases and veterinary parasitic diseases the present invention, will have important means of low cost production of vaccines in a form easily used for animal treatment.

Since many edible plants used by humans for food or as components of animal feed are dicotyledenous plants, it is preferred to employ dicotyledons in the present invention, although monocotyledon transformation is also applicable especially in the production of certain grains useful for animal feed.

The host plant selected for genetic transformation preferably has edible tissue in which the antigenic protein, a proteinaceous substance, can be expressed. Thus, the antigenic protein is expressed in a part of the plant, such as the fruit, leaves, stems, seeds, or roots, which may be consumed by a human or an animal for which the vaccine is intended. Although not preferred, a vaccine may be produced in a non-edible plant and administered by one of various other known methods of administering vaccines.

Various other considerations are made in selecting the host plant. It is sometimes preferred that the edible tissue of the host plant not require heating prior to consumption since the heating may reduce the effectiveness of the vaccine for animal or human use. Also, since certain vaccines are most effective when administered in the human or animal infancy period, it is sometimes preferred that the host plant express the antigenic protein which will function as a vaccine in the form of a drinkable liquid.

Plants which are suitable for the practice of the present invention include any dicotyledon and monocotyledon which is edible in part or in whole by a human or an animal such as, but not limited to, carrot, potato, apple, soybean, rice, corn, berries such as strawberries and raspberries, banana and other such edible varieties. It is particularly advantageous in certain disease prevention for human infants to produce a vaccine in a juice for ease of administration to humans such as tomato juice, soy bean milk, carrot juice, or a juice made from a variety of berry types. Other foodstuffs for easy consumption might include dried fruit.

Methods of Gene Transfer into Plants

There are various methods of introducing foreign genes into both monocotyledenous and dicotyledenous plants. The principle methods of causing stable integration of exogenous DNA into plant genomic DNA include the following approaches: 1) Agrobacterium—mediated gene transfer; 2) direct DNA uptake, including methods for direct uptake of DNA into protoplasts, DNA uptake induced by brief electric shock of plant cells, DNA injection into plant cells or tissues by particle bombardment, by the use of micropipette systems, or by the direct incubation of DNA with germinating pollen; or 3) the use of plant virus as gene vectors.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. The Agrobacterium system is especially viable in the creation of transgenic dicotyledenous plants.

As listed above there are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

The last principle method of vector transfer is the transmission of genetic material using modified plant viruses. DNA of interest is integrated into DNA viruses, and these viruses are used to infect plants at wound sites.

In the preferred embodiment of the present invention, the Agrobacterium—Ti plasmid system is utilized. The tumor-inducing (Ti) plasmids of A. tumefaciens contain a segment of plasmid DNA called transforming DNA (T-DNA) which is transferred to plant cells where it integrates into the plant host genome. The construction of the transformation vector system has two elements. First, a plasmid vector is constructed which replicates in Escherichia coli (E. coli). This plasmid contains the DNA encoding the protein of interest (an antigenic protein in this invention); this DNA is flanked by T-DNA border sequences that define the points at which the DNA integrates into the plant genome. Usually a gene encoding a selectable marker (such as a gene encoding resistance to an antibiotic such as Kanamycin) is also inserted between the left border (LB) and right border (RB) sequences; the expression of this gene in transformed plant cells gives a positive selection method to identify those plants or plant cells which have an integrated T-DNA region. The second element of the process is to transfer the plasmid from E. coli to Agrobacterium. This can be accomplished via a conjugation mating system, or by direct uptake of plasmid DNA by Agrobacterium. For subsequent transfer of the T-DNA to plants, the Agrobacterium strain utilized must contain a set of inducible virulence (vir) genes which are essential for T-DNA transfer to plant cells.

Those skilled in the art should recognize that there are multiple choices of Agrobacterium strains and plasmid construction strategies that can be used to optimize genetic transformation of plants. They will also recognize that A. tumefaciens may not be the only Agrobacterium strain used. Other Agrobacterium strains such as A. rhizogenes might be more suitable in some applications.

Methods of inoculation of the plant tissue vary depending upon the plant species and the Agrobacterium delivery system. A very convenient approach is the leaf disc procedure which can be performed with any tissue explant that provides a good source for initiation of whole plant differentiation. The addition of nurse tissue may be desirable under certain conditions. Other procedures such as the in vitro transformation of regenerating protoplasts with A. tumefaciens may be followed to obtain transformed plant cells as well.

This invention is not limited to the Agrobacterium-Ti plasmid system but should include any direct physical method of introducing foreign DNA into the plant cells, transmission of genetic material by modified plant viruses, and any other method which would accomplish foreign DNA transfer into the desired plant cells.

Promoters Once the host plant has been selected and the method of gene transfer into the plant determined, a constitutive, a developmentally regulated, or a tissue specific promoter for the host plant is selected so that the foreign protein is expressed in the desired part(s) of the plant.

Promoters which are known or found to cause transcription of a foreign gene in plant cells can be used in the present invention. Such promoters may be obtained from plants or viruses and include, but are not necessarily limited to: the 35S promoter of cauliflower mosaic virus (CaMV) (as used herein, the phrase “CaMV 35S” promoter includes variations of CaMV 35S promoter, e.g. promoters derived by means of ligations with operator regions, random or controlled mutagenesis, etc.); promoters of seed storage protein genes such as Zma10Kz or Zmag12 (maize zein and glutelin genes, respectively), light-inducible genes such as ribulose bisphosphate carboxylase small subunit (rbcS), stress induced genes such as alcohol dehydrogenase (Adh1), or “housekeeping genes” that express in all cells (such as Zmaact, a maize actin gene). This invention can utilize promoters for genes which are known to give high expression in edible plant parts, such as the patatin gene promoter from potato.

The plasmid constructed for plant transformation also usually contains a selectable or scorable marker gene. Numerous genes for this purpose have been identified.

Following transformation of target tissues, expression of the above-described selectable marker genes allows for preferential selection of transformed cells, tissues and/or plants, using regeneration and selection methods now well known in the art.

After transformation of a plant cell or plant, plant cells or plants transformed with the desired DNA sequences integrated into the genome can be selected by appropriate phenotypic markers. Phenotypic markers are known in the art and may be used in this invention.

Confirmation of transgenic plants will typically be based on an assay or assays or by simply measuring stress response. Transformed plants can be screened by biochemical, molecular biological, and other assays. Various assays may be used to determine whether a particular plant, plant part, or a transformed cell shows an increase in enzyme activity or carbohydrate content. Typically, the change in expression or activity of a transformed plant will be compared to levels found in wild type (e.g., untransformed) plants of the same type. Preferably, the effect of the introduced construct on the level of expression or activity of the endogenous gene will be established from a comparison of sibling plants with and without the construct. Protein levels can be measured, for example, by Northern blotting, primer extension, quantitative or semi-quantitative PCR (polymerase chain reaction), and other methods well known in the art (See, e.g., Sambrook, et al. (1989). Molecular Cloning, A Laboratory Manual, second edition (Cold Spring Harbor Laboratory Press), Vols. 1-3). Protein can be measured in a number of ways including immunological methods (e.g., by Elisa or Western blotting). Protein activity can be measured in various assays as described in Smith (Smith, A. M. (1990). In: Methods in Plant Biochemistry, Vol. 3, (Academic Press, New York), pp. 93-102).

Normally, regeneration will be involved in obtaining a whole plant from a transformation process. The term “regeneration” as used herein, means growing a whole plant from a plant cell, a group of plant cells, a plant part, or a plant piece (e.g., from a protoplast, calys, or a tissue part).

The foregoing methods for transformation would typically be used for producing transgenic inbred lines. Transgenic inbred lines could then be crossed, with another (non-transformed or transformed) inbred line, in order to produce a transgenic hybrid plant. Alternatively, a genetic trait which has been engineered into a particular line using the foregoing transformation techniques could be moved into another line using traditional backcrossing techniques that are well known in the plant breeding arts. For example, a backcrossing approach could be used to move an engineered trait from a public, non-elite line into an elite line, or from a hybrid plant containing a foreign gene in its genome into a line or lines which do not contain that gene. As used herein, “crossing” can refer to a simple X by Y cross, or the process of backcrossing, depending on the context.

Parts obtained from the regenerated plant, such as flowers, pods, seeds, leaves, branches, fruit, and the like are covered by the invention, provided that these parts comprise cells which have been so transformed. Progeny and variants, and mutants of the regenerated plants are also included within the scope of this invention, provided that these parts comprise the introduced DNA sequences.

Once a transgenic plant is produced having a desired characteristic, it will be useful to propagate the plant and, in some cases, to cross to inbred lines to produce useful hybrids.

In seed propagated crops, mature transgenic plants may be self crossed to produce a homozygous inbred plant. The inbred plant produces seed containing the genes for the newly introduce trait. These seeds can be grown to produce plants that will produce the selected phenotype which are then harvested and administered.

Methods of administering any of the immunologic compositions of the invention are also provided. In certain general embodiments, such methods comprise administering a protective amount of the composition to a mammal. In more specific embodiments, these methods entail oral introduction of the composition either into a mammalian subject. Traditionally those skilled in the art of vaccination would dose to achieve vaccination at the lowest dose possible in a dose-dependent manner and by so doing elicit serum and/or secretory antibodies against the immunogen of the vaccine with minimal induction of systemic tolerance. According to the invention, however dosing is accomplished at much lower levels, only sufficient to generate protection, without an antibody response. Methods for dosing and regulating antibody presence are known to those of skill in the art and determination of the appropriate dose consistent with the teachings herein amounts to nothing more that routine optimization of parameters. The course of the immunization may be followed by assays for antibodies for the supernatant antigens. The assays may be performed by labeling with conventional labels, such as radionuclides, enzymes, fluorescers, and the like. These techniques are well known and may be found in a wide variety of patents, such as U.S. Pat. Nos. 3,791,932; 4,174,384 and 3,949,064, as illustrative of these types of assays.

The following examples are offered to illustrate but not limit the invention. Thus, they are presented with the understanding that various formulation modifications may be made and are to be within the scope of the invention.

### EXAMPLE 1

Introduction

The heat-labile enterotoxins of Escherichia coli are a member of a family of bacterial toxins of oligomeric protein toxins composed of one A polypeptide and five B polypeptides maintained by noncovalent bonding in a quaternary structure between the A subunit and a pentameric ring of B subunits. (Spangler, B. D. 1992, Gill, D. M. et al, 1981, Hardy, S. J. et al, 1988, Sixma, T. K. et al, 1991). The Lt toxin has been shown extensively in the literature to possess strong adjuvant properties that stimulate mucosal IgA as well as systemic IgG immune responses when coadministered with an unrelated antigen. The mechanism of this adjuvanticity is under intense investigation. In particular administration of the Lt toxin has been shown to elevate levels of the immunostimulatory cytokines interferon λ (IFN-λ) and interleukin-4 (IL-4), important mediators in the IgG response. Elevated IFN-λ levels have been demonstrated to be due to the activity of the Lt B subunit while IL-4 elevated levels are due to the activity of the A subunit. (Martin, M. et al, 2000).

Diseases due to viral infections in animals result in considerable economic losses each year. In particular a number of virus infections have been shown to be sensitive to interferon-a levels. In one particular study, swine testis cell cultures pretreated with various does of recombinant human interferon alpha and infected with the swine virus, transmissible gastroenteritis virus, showed significantly reduced titers of virus infection relative to the controls (Jordan, L. T. et al, 1994, Jordan, L. T. et al, 1995, La Bonnardierre, C. et al, 1983, Weingartl, H. M. et al, 1991). This same effect has been shown with a number of other viruses including porcine respiratory and reproductive syndrome (PRRS) (Buddaert, W. et al, 1998, Albina, E. et al 1998) and rotaviruses (La Bonnardierre, C. et al 1983). This phemonem appears to be quite broad against a fairly wide range of viruses (Derbyshire, JB, 1991).

In this study, we orally dosed 13-day-old piglets for ten days with an Lt mutant deficient in subunit A activity and subsequently challenged with a virulent Purdue strain of TGEV. This group of piglets was protected from the disease as contrasted to the control group.

### MATERIALS AND METHODS

Lt Toxin

Mutant heat-labile enterotoxin of Escherichia coli (R192G) was obtained from John Clements, Ph.D. Tulane University. (5 mg of LT(R192G) in 1 ml of TEAN (0.05 M Tris, 0.001 M EDTA, ) 0.003 M NaN3, 0.2 M NaCl, pH 7.5). Adjuvant was rehydrated with 1 ml of sterile water and diluted 1:20 in sterile water and dispensed in 0.5 ml aliquots and stored at 4° C. This dilution represented a concentration of 25 μg's per 0.5 ml.

Swine Feeding Trial

Swine feeding trials were conducted as follows. 10-day-old SPF TGEV sero-negative pigs from a low disease incidence herd were utilized in this trial.

Treatment Groups

The study consisted of three treatment groups; Group A was fed Lt toxin and control corn and Group B was fed control corn. Table 1 shows a summary of the design of the study.

TABLE 1
Summary of Study Design

| | | Vaccine | | | | |
|---|---|---|---|---|---|---|
| | Number | Descrip- | Amount/ | | | Day of |
| Group | of pigs | tion | day | Route | Timing | Challenge |
| A | 10 | Lt toxin + | 25 μgs | Oral | 0 to 10 | Day 12 |
| | | control | of Lt | | days | |
| | | corn | toxin + | | | |
| | | | 50 grams | | | |
| | | | control | | | |
| | | | corn | | | |
| B | 10 | Control | 50 grams | Oral | 0 to 10 | Day 12 |
| | | corn | control | | days | |
| | | | corn | | | |

Treatment of Feed Test Groups

For 10 consecutive days, all piglets were withheld from feed overnight and all feed-test groups were treated first thing in the morning. In Group A, 25 micrograms of Lt toxin in 0.5 ml was needed per day per pig. Medicated milk replacer a total of not less than 300 ml and not more than 600 ml was used as a base to which the ground corn was added and mixed so as to produce a thick oatmeal-type meal. The corn was stirred in with a clean wooden stick until thick with just a little milk settling to the top. This amounted to approximately 1000 grams of feed representing 100 grams per piglet feeding. A line of meal was placed on a clean dry floor and the piglets allowed to consume the vaccine. Attempts were made to ensure each piglet received an adequate portion. After the ration was consumed, regular water and medicated weaning rations were replaced in the pen.

Group A (Lt Toxin +Control Corn 50 Gram Dose):

Prior to adding control corn all animals in this group received an individual 0.5 ml oral dose (25 μg's) of E. coli LT adjuvant. The piglets were then allowed to consume the control corn feed which was composed of 500 grams of control corn and 300-600 ml of medicated milk replacer. This was hydrated with water and fed to each piglet for 10 consecutive days, first thing in the morning representing approximately 100 grams per piglet feeding.

Group B (Control Corn, 50 Gram Dose):

The piglets were allowed to consume the control corn feed which was composed of 500 grams of control corn and 300-600 ml of medicated milk replacer. This was hydrated with water and fed to each piglet for 10 consecutive days, first thing in the morning representing approximately 100 grams per piglet feeding.

Virus Challenge

On day 12 (2 days after last feed vaccination and 5 days after MLV TGEV) all animals were orally challenged with a 2 ml oral dose of virulent TGEV challenge (Purdue strain, titer of 10^{7.6} FAID₅₀'s per dose). Previous work had determined that this challenge strain and level would produce a clinically typical TGEV watery diarrhea in 21 to 28 day old piglets that will persist for 7 to 10 days. No mortality values have been observed with this challenge model in this age animal.

Data and Sample Collection

Persons performing daily observations were blinded as to treatment.

1) Daily Observations: Piglets were observed twice daily and for any signs of diarrhea and scored as below:

0 (Normal)

2 (Creamy, piles up in pen)

4 (Watery)

Additional clinical signs which was observed such as dehydration or depression, anorexia, vomitus and death were scored as below and the number added to fecal observation for a total clinical score as shown below. Any animal that died or appeared moribund was sacrificed and necropsied. A sample from the jejunum of the small intestine was collected and observed for villous atrophy and providing that the sample was not too necrotic it was assayed for TGEV. Attempts were made to isolate TGEV from the feces of watery scouring animals so as to confirm the challenge. A fecal sample was collected and TGEV isolation was conducted by inoculating confluent ST cells and staining by specific immunofluorescence.

1 (Dehydration & Depression)

1 (Anorexia)

3 (Vomitus)

10 (Moribund or Death)

2) Weights: All animals were weighed on day 0, day 12 and day 24.

3) Blood Samples: Blood were collected on day 0, day 12 and day 26. Blood was allowed to clot and serum collected and stored at 20° C. until assay. Sera was assayed for TGEV neutralizing titers and titer values calculated using a Spermen Karber table.

4) Fecal Samples: Fecals were collected from randomly selected animals within a group that showed watery diarrhea and fecals were checked for TGEV activity.

Data Analysis

The total clinical scores for all animals within their group was divided by the number of observations to give a group clinical score. Statistical differences between groups were compared. The clinical symptom data are presented as a Percent Morbidity Incidence, (number of animals with clinical signs >2 divided by total number of animals); Percent Morbidity Incidence and Duration (total number of clinical observations >2 divided by total number of pig days) and a Clinical Severity Index (Total clinical score value divided by total number of pig days).

### RESULTS

A. Clinical Symptoms Observations

Table 2: Post-Challenge Group Morbidity Values and Percent Reductions. Percent Morbidity Incidence is defined as the number of animals with clinical signs >2 divided by total number of animals. Percent Morbidity Incidence and Duration is defined as the number of animals with clinical signs >2 divided by total number of clinical observations >2 divided by total number of pig days.

| | | Morbidity Incidence & | | |
|---|---|---|---|---|
| | | Duration (Percent Reduction) | | |
| | Morbidity | 1-DPC to | 8-DPC to | 1-DPC to |
| Group | Incidence | 7-DPC | 14-DPC | 14-DPC |
| A: | 50% | 29% | 3% | 15% |
| Lt mutant | | | | |
| and control | | | | |
| corn | | | | |
| B: | 100% | 59% | 6% | 31% |
| Control | | | | |
| Corn | | | | |

Group A which received the Lt mutant were significantly protected from the virulent challenge of the virus compared to the controls. All pigs in Group B developed significant disease symptoms (morbidity) while only a portion of those in Group A developed any symptoms.

TABLE 3
Clinical Severity Index Summaries and
Percent Reductions. Clinical Severity
Index is defined as the total clinical
score value divided by total number of pig days.

| | Clinical Severity Index (Percent Reduction) | | |
|---|---|---|---|
| Group | 1-DPC to 7-DPC | 8-DPC to 14-DPC | 1-DPC to 14-DPC |
| A: | | | |
| Lt mutant and | 0.89 | 0.20 | 0.54 |
| control corn | | | |
| B: | | | |
| Control Corn | 2.04 | 0.19 | 1.11 |

When the observed clinical symptoms are rated with a clinical severity index, the pigs receiving an oral dose of the Lt toxin were significantly protected from a virulent challenge of the TGEV virus.

B. Virus Isolation Fecal samples were collected from each pig at 3 and 6 days post-challenge. TGEV was isolated from fecal samples collected from pigs in each group confirming that the diarrhea that was being observed was due to the TGEV challenge. The highest incidence of TGEV isolation occurred in the animals that were fed control corn (group B) but was not at a significantly greater rate than the isolation values seen in the other groups. It should be noted that isolation of TGEV from infected animals is variable and virus isolation and rate of shedding does not correlate well to the amount of protection or susceptibility. Rather the important aspect of the TGEV isolation is to confirm that the clinical signs observed were due to the TGEV challenge.

Animals orally dosed with Lt mutant toxin were protected from a virulent challenge of the virus. Moreover these observations also extend to weight gain of these animals since the group receiving the Lt mutant toxin also gained weight relative to the corn control. Thus oral dosage of Lt toxin or the Lt B subunit will protect against viral infection and in the case of food animals the oral dosage can also be used as a method to increase weight gain and overall weight. This level of protection seen in this study includes general health and vigor, a decrease in clinical symptoms, lack of virus shedding and other observations known to be criteria for disease protection. The mechanism of protection is unknown but may be an active immune response by the animal or interference with parts of the viral replicative process. This result is surprising in that while Lt has been shown to elevate levels of the cytokine INF-λ, viruses are sensitive to elevated levels of the cytokine interferon—α. However in this study administration of the Lt results in virus protection and increased weight gain. Oral administration of Lt toxin or one of its subunits to animal will protect the animal from disease and increase weight gain of the animal.

### REFERENCE LIST

Albina, E., Carrat, C., and Bernard Charley. 1998. Interferon-α response to swine arterivirus (PoAV), the Porcine Reproductive and Respiratory Syndrome Virus. Journal of Interferon and Cytokine Research 18:485-490.

La Bonnardière, C. and H. Laude. 1 983. Interferon induction in rotavirus and coronavirus infections: A review of recent results. Ann. Rech. Vèt., 14:507-511.

Buddaert, W., K. Van Reeth and M. Pensaert. 1998. In vivo and in vitro interferon (IFN) studies with the porcine reproductive and respiratory syndrome virus (PRRSV). Coronaviruses and Arteriviruses, edited by Enjuanes et al., Plenum Press 59:461-467.

Derbyshire, J. B. 1989. The interferon sensitivity of selected porcine viruses. Can J. Vet Res 53:52-55.

Gill, D. M., J. D. Clements, D. C. Robertson, and R. A. Finkelstein. 1981. Subunit number and arrangements in Escherichia coli heat-labile enterotoxin. Infect. Immun. 56:1748-1753.

Hardy, S. J., J. Holmgren, S. Johansson, J. Sanchez and T. R. Hirst. 1988. Coordinated assembly of multisubunit proteins: oligomerization of bacterial enterotoxins in vivio and in vitro. Proc. Natl. Acad. Sci. USA 85: 7109-7113.

Jordan, L. T., and J. B. Derbyshire. 1994. Antiviral activity of interferon against transmissible gastroenteritis virus in cell culture and ligated intestinal segments in neonatal pigs. Veterinary Microbiology, Elsevier Science B.V., Amsterdam 38:263-276.

Jordan, L. T. and J. B. Derbyshire. 1995. Anitviral action of interferon-alpha against porcine transmissible gastroenteritis virus. Veterinary Microbiology 45:59-70.

Martin, Michael, Daniel J. Metzger, Suzanne M. Michalek, Terry D. Connell, and Michael W. Russell. 2000. Comparative analysis of the mucosal adjuvanticity of the Type II heat-labile enterotoxins LT-IIa and LT-IIb. Infection and Immunity 68(1):281-287.

Sixma, T. K., S. E. Pronk, K. H. Kalk, E. S. Wartna, B. A. van Zanten,B. Witholt, and W. G. Hol. 1991. Crystal structure of a cholera toxin-related heat-labile enterotoxin from E. coli. Nature 351:371-377.

Spanger, B. D. 1992 Structure and function of cholera toxin and the related Eschericia coli heat-labile enterotoxins. Microbiol. Rev. 56:622-647.

Weingartl, Hana Al. and J. Brian Derbyshire. 1991. Antiviral activity against transmissible gastroenteritis virus, and cytotoxicity, of natural porcine interferons alpha and beta. Can J Vet Res 55:143-149.

### EXAMPLE 2

In this example, we orally dosed 12-14 day old piglets for eight days with an Lt mutant deficient in subunit A activity and subsequently challenged with a virulent Purdue strain of TGEV on Day 18 of the study. Unless indicated all conditions in this example are the same as those given in Example 1.

### MATERIALS AND METHODS

Treatment Groups

The study consisted of two treatment groups: Group A was fed Lt toxin +control corn and Group B was fed control corn. Table 2 shows a summary of the design of the study.

TABLE 4
Summary of Study Design

| | | Vaccine | | | | |
|---|---|---|---|---|---|---|
| | Number | Descrip- | Amount/ | | | Day of |
| Group | of pigs | tion | day | Route | Timing | challenge |
| A | 10 | Lt toxin + | 25 ugs | Oral | 0 to 8 | Day 18 |
| | | control | of Lt | | days | |
| | | corn | toxin + | | | |
| | | | 5o grams | | | |
| | | | control | | | |
| | | | corn | | | |
| B | 10 | Control | 50 grams | Oral | 0 to 8 | Day 18 |
| | | corn | control | | days | |
| | | | corn | | | |

Treatment of Test Groups

Identical to Example 1 except that Group A and Group B were treated with the appropriate treatment for eight days in contrast to Example 1 in which they were treated for 10 days.

Virus Challenge

Identical to Example 1 except all animals were challenged on Day 18 of the study.

### RESULTS

A. Clinical Symptoms Observations

Clinical signs typical of TGEV infection with severe watery diarrhea were not as frequent as in Example 1. This is attributed to the age of the animals at time of challenge. The animals were 30-32 days of age at time of challenge and TGEV infections in older animals (>28 days of age) are less severe than in animals at time of challenge.

TABLE 5
Post-Challenge Group Morbidity Values and
Percent Reductions. Percent Morbidity Incidence is
defined as the number of animals with clinical
signs >2 divided by total number of animals.
Percent Morbidity Incidence and Duration is
defined as the number of animals with clinical
signs ≧2 divided by total number of clinical
observations ˜2 divided by total number of pig days.

| | | Morbidity Incidence & Duration |
|---|---|---|
| | Morbidity | (Percent Reduction) |
| Group | Incidence | 1-DPC to 10-DPC |
| A: | 27% | 4% |
| Lt mutant | | |
| and control corn | | |
| B: | 50% | 13% |
| Control Corn | | |

Group A which received the Lt mutant were significantly protected from the virulent challenge of the virus compared to the controls. More pigs in Group B developed significant disease symptoms (morbidity) compared to those in Group A.

TABLE 6
Clinical Severity Index Summaries and
Percent Reductions. Clinical Severity Index
is defined as the total clinical score value
divided by total number of pig days.

| | Clinical Severity Index |
|---|---|
| | (Percent Reduction) |
| Group | 1-DPC to 10-DPC |
| A: | 0.07 |
| Lt mutant | |
| and control corn | |
| B: | 0.36 |
| Control Corn | |

When the observed clinical symptoms are rated with a clinical severity index, the pigs receiving an oral dose of the Lt toxin were significantly protected from a virulent challenge of the TGEV virus.

FIG. 4 shows that 50% of the pigs that were fed control corn developed TGEV clinical symptoms, while only 27% of those that received the Lt exhibited symptoms.

FIG. 5 shows that pigs that were fed cintrol corn developed TGEV clinical symptoms while those that received the control Lt exhibited fewer symptoms.

FIG. 6 shows that the disease that developed in the pigs that were fed control corn was much higher than the Lt group.

B. Virus Isolation

Fecal samples were collected from each pig at 3 and 6 days post-challenge. TGEV was isolated from fecal samples collected from pigs in each group confirming that the diarrhea that was being observed was due to the TGEV challenge. The rate of TGEV isolation from the fecals was greater at 3 days than at 6 days post-challenge see addendum's 2 through 6. The highest incidence of TGEV isolation occurred in the animals that were fed control} corn (group C) but was not at a significantly greater rate than the isolation values seen in the other groups. It should be noted that isolation of TGEV from infected animals is variable and virus isolation and rate of shedding ˜does not correlate well to the amount of protection or susceptibility. Rather the important aspect of the TGEV isolation is to confirm that the clinical signs observed were due to the TGEV challenge.

### EXAMPLE 3

Swine transmissible gastroenteritis (TGE) (Saif, L. J. et al., 1992) is recognized as one of the major causes of sickness and death in piglets particularly in areas with high concentrations of pigs. TGE is a highly contagious enteric disease that is characterized by vomiting, severe diarrhea and high mortality in piglets less than two weeks of age. The causal agent of TGE is a pleomorphic, enveloped single-stranded RNA virus belonging to the genus Coronavirus of the family Coronaviridae. Replication of virus in the villous epithelial cells of the small intestine results in the destruction or alteration of function of these cells. These changes lead to a reduction in the activity of the small intestine that disrupts digestion and cellular transport of nutrients and electrolytes. In small piglets this can lead to a severe and fatal deprivation of nutrients and dehydration. Following infection, pigs that have survived the infection are immune to subsequent infections presumably due to local immunity in the intestinal mucosa. Thus, since active immunity towards TGEV involves local immunity in the intestinal mucosa, presumably through the activation and secretion of intestinal SIgA, vaccines that target activation of the intestinal mucosa immune system are particularly attractive in the control of this disease. In particular, the development of edible vaccines offers the potential to aid in the control of enteric diseases such as TGE. Edible vaccines from plant material could be directly delivered in the feed and could be produced cheaply in large volumes thus avoiding many costs associated with the administration of conventional vaccines. Vaccines from plants are particularly suitable for stimulation of mucosal immunity, since edible plant products can be delivered orally to reach the gut mucosal tissue and elicit an immune response at mucosal surfaces. Recent advances in technology make it now possible to express vaccine antigens at high levels in plants.

A number of different plant systems have recently been under investigation for use in edible oral delivery systems. Of these, the system based on the use of transgenic maize seed appears to be the most realistic for a number of different reasons. Among these reasons include the ability to introduce a grain-based product directly into a producer's feed system, the ability to utilize the already existing infrastructure for the production, harvesting, transportation, storage, and processing of the grain, the ability to deliver a product (both monovalent and multivalent) at a cost competitive with contemporary vaccines due to a low cost of goods, and a plant system amenable to transformation with highly developed and characterized genetics.

TGEV virions contain three major structural proteins: a nucleocapsid protein (N), a small membrane-bound glycoprotein (M), and large spike or peplomer glycoprotein (S). In this study, we generated transgenic maize plants that express the spike protein at high levels. Corn expressing the S protein of TGEV was fed to 13-day-old piglets for ten days and subsequently challenged with a virulent Purdue strain of TGEV. This group of piglets was significantly protected from the disease as contrasted to the control group that was fed only corn. Results from a second trial duplicated these results demonstrating that the delivery of antigens delivered in an edible oral form are efficacious. Surprisingly pigs fed these plants were protected from challenge with TGEV in the absence of antibodies.

### MATERIALS AND METHODS

Construction of Plasmids used for Transformation and Agrobacterium-mediated Maize Transformation

The amino acid sequences of the various structural proteins of TGEV were back-translated using the Backtranslate program of the Wisconsin GCG Package against a codon table tabulated for highly expressed maize genes. The resulting DNA sequence was scanned for the presence of undesirable sequence, e.g. polyadenylation signals, 5′ and 3′ consensus splice sites, other mRNA destabilizing sequences, and undesirable endonuclease restriction enzyme sites. The DNA sequence was modified to eliminate these sites by choosing alternative codons. Alternative codons with a codon frequency of less than 10 percent for that amino acid were avoided. The resulting sequence was then constructed using a series of synthesized overlapping complementary oligonucleotides and the polymerase chain reaction (PCR) to amplify the resulting synthetic sequence. Convenient restriction sites were also engineered into the 5′ and 3′ ends of the optimized gene to facilitate cloning. The barley α-amylase signal sequence (Rogers J. C., 1985). Two barley alpha-amylase gene families are regulated differently in aleurone cells. J Biol Chem 260:3731-3738 (1985)) was also synthesized using overlapping complimentary nucleotides with maize-preferred codons.

Maize Transformation

Transgenic maize plants were generated using the method of Ishida, Y. et al., 1996. Essentially, maize corn ears were harvested at 9-12 days after pollination when embryos are approximately 1-2 mm in length. Whole ears were surface sterilized in 50% bleach (+teaspoon of Tween 20) for 30 min and given two rinses of sterile H₂O. Immature zygotic embryos (ZE) were sterilely isolated from the ears. Embryos were washed twice with co-cultivation medium and Agrobacterium was added directly by pouring bacterial solution into the ZE tube. Embryos with bacteria were vigorously vortexed for 30 seconds and allowed to incubate at room temperature for 5 minutes. Embryos were placed scutellum side up onto co-cultivation medium and incubated at 19° C. in the dark for 3-5 days. Keeping scutellum side up, embryos were transferred to antibiotic-containing medium without selection for three days in the dark at 27-28° C. every subsequent 2 weeks, embryos and herbicide resistant calli were transferred to fresh selection medium. When sufficient callus from a single event had developed on selection medium (approximately two plates), the callus was transferred onto regeneration medium. Mature somatic embryos were placed in the light and allowed to germinate. Ten plants from each event were transplanted to soil in the greenhouse and allowed to flower and produce seed. The resulting seed (Ti seed) was screened by ELISA to determine the levels of the recombinant protein of interest.

Transgenic Grain Production

Highly expressing seeds were backcrossed into maize lines of commercial interest. For this study, pollen from T1 seed was crossed to commercial maize hybrids in order to bulk up the seed as fast as possible. The resulting grain was ground to cornmeal (600-micron particle size). The levels of S protein in this fraction were estimated to be 0.004% (w/w). Piglets were fed about 50 grams per day of transgenic corn. That roughly amounted to 2 mg of S protein per dose per day.

Swine Feeding Trials

Swine feeding trials were conducted, and 10-14 day-old SPF TGEV sero-negative pigs from a low disease incidence herds were utilized in these trials.

Vaccination of Feed Test Groups

For the appropriate consecutive days, all piglets were withheld from feed overnight (including the MLV vaccinates) and all feed-test groups will be vaccinated first thing in the morning. In groups receiving the TGEV-S corn, 50 grams of TGEV transgenic corn was needed per day per pig. The dry corn was mixed with a wooden stick to ensure distribution of the transgenic corn. Medicated milk replacer a total of not less than 300 ml and not more than 600 ml was used as a base to which the ground corn was added and mixed so as to produce a thick oatmeal-type meal. The corn was stirred in with a clean wooden stick until thick with just a little milk settling to the top. This amounted to approximately 1000 grams of feed representing 100 grams per piglet feeding, containing 50 grams of transgenic corn per pig feeding. A line of vaccine meal was placed on a clean dry floor and the piglets allowed to consume the vaccine. Attempts were made to ensure each piglet received an adequate vaccine portion. After vaccine was consumed, regular water and medicated weaning rations were replaced in the pen. Pigs in the treatment group receiving the modified live vaccine (MLV) were orally vaccinated with MLV TGEV according to label directions at day 0 & 7 days later.

Virus Challenge

In the case of TGEV-1, on day 12 (2 days after last feed vaccination and 5 days after MLV TGEV) all animals were orally challenged with a 2 ml oral dose of virulent TGEV challenge (Purdue strain, titer of 10^{7.6} FAID₅₀'s per dose). Animals in TGEV-2, On day 18 (2 days after last feed vaccination for the 16-day groups and 11 days after MLV TGEV) all animals were orally challenged with a 2 ml oral dose of virulent TGEV challenge (Purdue strain, titer of 10^{7.6} FAID₅₀'s per dose). Previous work has determined that this challenge strain and levels will produce a clinically typical TGEV watery diarrhea in 21 to 28 day old piglets that persists for 7 to 10 days. No mortality values have been observed with this challenge model in this age animal.

Data and Sample Collection

Persons performing daily observations were blinded as to treatment.

1) Daily Observations: Piglets were observed twice daily and for any signs of diarrhea and scored as below:

0 (Normal)

2 (Creamy, piles up in pen)

4 (Watery)

Additional clinical signs which was observed such as dehydration or depression, anorexia, vomitus and death were scored as below and the number added to fecal observation for a total clinical score as shown below. Any animal that died or appeared moribund was sacrificed and necropsied. A sample from the jejunum of the small intestine was collected and observed for villous atrophy and providing that the sample was not too necrotic it was assayed for TGEV. Attempts were made to isolate TGEV from the feces of watery scouring animals so as to confirm the challenge. A fecal sample was collected and TGEV isolation was conducted by inoculating confluent ST cells and staining by specific immunofluorescence.

1 (Dehydration & Depression)

1 (Anorexia)

3 (Vomitus)

10 (Moribund or Death)

2) Weights: All animals were weighed on day 0, day 12 and day 24.

3) Blood Samples: Blood were collected on day 0, day 12 and day 26. Blood was allowed to clot and serum collected and stored at 200 C until assay. Sera was assayed for TGEV neutralizing titers and titer values calculated using a Spermen Karber table.

4) Fecal Samples: Fecals were collected from randomly selected animals within a group that showed watery diarrhea and fecals were checked for TGEV activity.

Data Analysis

The total clinical scores for all animals within their group was divided by the number of observations to give a group clinical score. Statistical differences between groups were compared. The clinical symptom data are presented as a Percent Morbidity Incidence, (number of animals with clinical signs >2 divided by total number of animals); Percent Morbidity Incidence and Duration (total number of clinical observations >2 divided by total number of pig days) and a Clinical Severity Index (Total clinical score value divided by total number of pig days).

Swine Feeding Trial #1 (TGEV-1)

Treatment Groups

The study consisted of three treatment groups; Group A was fed transgenic corn expressing the spike protein (S) of TGEV, Group B was fed non-transgenic corn, and Group C was vaccinated with a commercial modified live TGEV vaccine (MLV TGEV). Table 3 shows a summary of the design of the study.

TABLE 3
Summary of Study Design

| | | Vaccine | | | | |
|---|---|---|---|---|---|---|
| | Number | Descrip- | Amount/ | | | Day of |
| Group | of pigs | tion | day | Route | Timing | challenge |
| A | 10 | TGEV | 50 grams | Oral | 0 to 10 | Day 12 |
| | | transgenic | | | days | |
| | | corn | | | | |
| B | 10 | Control | 50 grams | Oral | 0 to 10 | Day 12 |
| | | corn | | | days | |
| C | 10 | MLV | N.A. | oral | 0 & 7 | Day 12 |
| | | TGEV | | | days | |

Swine Feeding Trial #2 (TGEV-2)

The study consisted of four treatment groups; Group A was fed transgenic corn expressing the spike protein (S) of TGEV, Group B was fed non-transgenic corn, and Group C was vaccinated with a commercial modified live TGEV vaccine (MLV TGEV). Table 4 shows a summary of the design of the study.

TABLE 4
Summary of Study Design

| | | Vaccine | | | | |
|---|---|---|---|---|---|---|
| | Number | Descrip- | Amount/ | | | Day of |
| Group | of pigs | tion | day | Route | Timing | challenge |
| A | 10 | TGEV | 50 grams | Oral | 0 to 4 | Day 18 |
| | | transgenic | | | days | |
| | | corn | | | | |
| B | 10 | TGEV | 50 grams | Oral | 0 to 8 | Day 18 |
| | | transgenic | | | days | |
| | | corn | | | | |
| C | 10 | TGEV | 50 grams | Oral | 0 to 16 | Day 18 |
| | | transgenic | | | days | |
| | | corn | | | | |
| D | 10 | Control | 50 grams | Oral | 0 to 16 | Day 18 |
| | | corn | | | days | |
| E | 10 | MLV | N.A. | Oral | 0 & 7 | Day 18 |
| | | TGEV | | (0.5 | days | |
| | | | | ml) | | |

### RESULTS

### Clinical Symptoms Observations TGEV-1

TABLE 5

| Treatment | Morbidity | Morbidity Incidence | Clinical Severity |
|---|---|---|---|
| Group | Incidence | and Duration | Index |
| TGEV corn | 50% | 20% | 0.83 |
| control corn | 100% | 31% | 1.11 |
| MLV TGEV | 89% | 16% | 0.78 |

Morbidity Incidence

FIG. 4 is a chart showing the percent of morbidity incidence in each of the treatment groups. This data is compilation of the significant (>2) clinical scores for each treatment group. This data shows that 100% of the pigs that were fed control corn developed TGEV clinical symptoms. Only 50% of those that received the TGEV corn exhibited symptoms and 89% of the pigs receiving the modified live vaccine developed symptoms.

Morbidity Incidence and Duration

FIG. 5 is a chart showing the percent of morbidity incidence and duration in each of the treatment groups. This data is compilation of the significant (>2) clinical scores for each treatment group. This data shows that the pigs that were fed control corn received a score of 31% while those that received the TGEV corn or those pigs receiving the modified live vaccine received scores of 20% and 16%, respectively.

Clinical Severity Index

FIG. 6 is a chart showing the clinical severity index for each treatment group. This data is compilation of the total clinical value divided by the total number of pig days for each treatment group. This data shows that the disease that developed in the pigs that were fed control corn was much higher than either the TGEV-S containing transgenic corn or the modified live vaccine treatment group.

Clinical Symptoms Observations

TGEV-2

Clinical signs typical of TGEV infection with severe watery diarrhea were not as frequent as in Example 1. This is attributed to the age of the animals at time of challenge. The animals were 30-32 days of age at time of challenge and TGEV infections in older animals (>28 days of age) are less severe than in animals at time of challenge.

TABLE 5

| | | | Percent | Percent Morbidity | Clinical |
|---|---|---|---|---|---|
| Treatment | Dose | | Morbidity | Incidence and | Severity |
| Group | Duration | | Incidence | Duration | Index |
| TGEV corn | 4 | days | 0 | 0 | 0 |
| | 8 | days | 20 | 5 | 0.16 |
| | 16 | days | 36 | 5 | 0.15 |
| control corn | 16 | days | 50 | 13 | 0.36 |

| | | | | |
|---|---|---|---|---|
| MLV TGEV | NA | 9 | 2 | 0.05 |

Morbidity Incidence

FIG. 6 is a chart showing the percent of morbidity incidence in each of the treatment groups. This data is compilation of the significant (>2) clinical scores for each treatment group. This data shows that 50% of the pigs that were fed control corn developed TGEV clinical symptoms. 0%, 20% and 36% of the pigs that received 4 days, 8 days and 16 days of TGEV corn, respectively, while 9% of the pigs receiving the modified live vaccine developed symptoms.

Morbidity Incidence and Duration

FIG. 7 is a chart showing the percent of morbidity incidence and duration in each of the treatment groups. This data is compilation of the significant (>2) clinical scores for each treatment group.

Clinical Severity Index

FIG. 8 is a chart showing the clinical severity index for each treatment group. This data is compilation of the total clinical value divided by the total number of pig days for each treatment group. This data shows that the disease that developed in the pigs that were fed control corn was much higher than either the TGEV-S containing transgenic corn or the modified live vaccine treatment group.

Table 1 and Table 2 show the seroconversion of serum in the animals in both tests (TGEV-I and TGEV-2). Values are shown as mean titers (GMT) from a TGEV neutralization assay. As can be shown in Table 1 and Table 2, those animals fed TGEV corn did not develop scrum antibodies levels and were identical to the control corn group.

TABLE 1
TGEV-1

| | TGEV Corn | Control Corn | MLV TGEV |
|---|---|---|---|
| Day of Treatment | (GMT) | (GMT) | (GMT) |
| 0 | <2 | <2 | <2 |
| 11 (challenge) | 2.5 | 2.2 | 12 |
| 28 (poss challenge) | 488 | 669 | 520 |

TABLE 2
TGEV-2

| | TGEV Corn | Control Corn | MLV TGEV |
|---|---|---|---|
| Date | (GMT) | (GMT) | (GMT) |
| 1 | <2 | <2 | <2 |
| 18 (challenge) | <2 | <2 | 12 |
| 29 (poss challenge) | 38 | 78 | 360 |

### DISCUSSION

Over the past decade, transgenic plants have been successfully used to express a variety of genes from bacterial and viral pathogens. Many of the resulting peptides induced an immunologic response in mice (Gomez, N. et al., 1998; Mason, H. S. et al.,1998; Wigdorovitz, A. et al, 1999) and humans (Kapusta, J. et al., 1999) comparable to that of the original pathogen. Characterization studies of these engineered immunogens have proven the ability of plants to express, fold and modify proteins in a manner that is consistent with the authentic source.

Numerous genes have been cloned into a variety of transgenic plants including many enzymes that have demonstrated the same enzymatic activity as their authentic counterparts (Hood, E. E. et al., 1997; Moldoveanu, Z. et al., 1999; Trudel, J. et al., 1992). Many additional genes have been expressed in plants solely for their immunogenic potential, including viral proteins (Gomez, N. et al., 1998; Kapusta, J. et al., 1999; Mason, H. S. et al., 1996; McGarvey, P. B. et al., 1995; Thanavala, Y. et al., Wigdorovitz, A. et al., 1999) and subunits of bacterial toxins (Arakawa, T. et al., 1997; Arakawa, T. et al., 1999; Haq, T. et al., 1995; Mason, H. S. et al., 1998). Animal and human immunization studies have demonstrated the effectiveness of many plant-derived recombinant antigens in stimulating the immune system. The production of antigen-specific antibodies and protection against subsequent toxin or pathogen challenge demonstrates the feasibility of plant derived-antigens for immunologic use.

The utilization of transgenic plants for vaccine production has several potential benefits over traditional vaccines. First, transgenic plants are usually constructed to express only a small antigenic portion of the pathogen or toxin, eliminating the possibility of infection or innate toxicity and reducing the potential for adverse reactions. Second, since there are no known human or animal pathogens that are able to infect plants, concerns with viral or prion contamination are eliminated. Third, immunogen production in transgenic crops rely on the same established technologies to sow, harvest, store, transport, and process the plant as those commonly used for food crops, making transgenic plants a very economical means of large-scale vaccine production. Fourth, expression of immunogens in the natural protein-storage compartments of plants maximizes stability, minimizes the need for refrigeration and keeps transportation and storage costs low (Kusnadi, J. et al., 1998; Kusnadi, A. R. et al., 1998). Fifth, formulation of multicomponent vaccines is possible by blending the seed of multiple transgenic corn lines into a single vaccine. Sixth, direct oral administration is possible when immunogens are expressed in commonly consumed food plants, such as grain, leading to the production of edible vaccines.

Some of the first attempts to make edible vaccines included transgenic potatoes expressing the E. coli heat-labile enterotoxin (LT-B) (Haq, T. A. et al., 1995), and a Norwalk virus surface protein (Mason, H. S. et al., 1996). In both cases, mice fed the antigenic tubers produced serum and secretory antibodies specific to the authentic antigen. Subsequently, many plant-expressed antigens, including those referenced above, have been shown to elicit an immune response when administered through an oral route. Several of these antigens have shown sufficient promise to warrant human clinical trials (Mason, H. S. et al., 1998; Saif, L. J. et al., 1994).

One of the most promising aspects of edible vaccines is the ability of orally administered immunogens to stimulate a mucosal immune response (Ruedl, C. et al., 1995). Mucosal surfaces, the linings of the respiratory, gastrointestinal, and urogenital tracts, play an important physical and chemical role in protecting the body from invading pathogens and harmful molecules. The mucosal immune system is distinct and independent of the systemic, or humoral, immune system, and is not effectively stimulated by parenteral administration of immunogens (Czerkinsky, C. et al., 1993). Rather, the mucosal immune system requires antigen presentation directly upon the mucosal surfaces. Since most invading pathogens first encounter one or more of the mucosal surfaces stimulation of the mucosal immune system is often the best first defense against many transmissible diseases entering the body through oral, respiratory and urogenital routes (Holmgren, J. et al., 1994).

Transgenic plants could produce large quantities of immunologically active recombinant antigen, very economically, for vaccine production. Multicomponent vaccines could easily be formulated from the seed of multiple transgenic plant lines to generate an increased chance for successful virus neutralization, in a stand-alone vaccination strategy, as a booster, or in combination with other vaccines and vaccination routes.

We report the protection of an economically important animal from a naturally occurring disease by an oral vaccination using an edible system in which no antibody response is observed. Moreover this system uses the conventional feed materials, e.g. corn, to deliver the antigen. One report (Modelska, A. et al., 1998) has shown in the laboratory the amelioration of rabies symptoms in mice fed multiple doses of a chimeric plant virus expressing the rabies glycoprotein following challenge with an attenuated rabies strain. The level of protection seen in this study includes general health and vigor, a decrease in clinical symptoms, lack of virus shedding and other observations known to be criteria for disease protection.

### REFERENCE LIST

Ahmad, S., B. Lohman, M. Marthas, L. Giavedoni, Z. el Amad, N. L. Haigwood, C. J. Scandella, M. B. Gardner, P. A. Luciw, and T. Yilma. 1994. Reduced virus load in rhesus macaques immunized with recombinant gp160 and challenged with simian immunodeficiency virus. AIDS Res. Hum. Retroviruses 10:195-204.

Arakawa, T., D. K. Chong, J. L. Merritt, and W. H. Langridge. 1997. Expression of cholera toxin B subunit oligomers in transgenic potato plants. Transgenic Res. 6:403-413.

Arakawa, T., J. Yu, and W. H. Langridge. 1999. Food plant-delivered cholera toxin B subunit for vaccination and immunotolerization. Adv. Exp. Med. Biol. 464:161-178.

Bergmeier, L. A., E. A. Mitchell, G. Hall, M. P. Cranage, N. Cook, M. Dennis, and T. Lehner. 1998. Antibody-secreting cells specific for simian immunodeficiency virus antigens in lymphoid and mucosal tissues of immunized macaques. AIDS 12:1139-1147.

Czerkinsky, C., A. M. Svennerholm, and J. Holmgren. 1993. Induction and assessment of immunity at enteromucosal surfaces in humans: implications for vaccine development. Clin. Infect. Dis. 16 Suppl 2:S106-S116.

Gomez, N., C. Carrillo, J. Salinas, F. Parra, M. V. Borca, and J. M. Escribano. 1998. Expression of immunogenic glycoprotein S polypeptides from transmissible gastroenterities coronavirus in transgenic plants. Virology 249:352-358.

Guidry, J. J., L. Cardenas, E. Cheng, and J. D. Clements. 1997. Role of receptor binding in toxicity, immunogenicity, and adjuvanticity of Eschericia coli heat-labile enterotoxin. Immunology 65:4943-4950.

Haq, T. A., H. S. Mason, J. Clements, and C. J. Arntzen. 1995. Production of an orally immunogenic bacterial protein in transgenic plants: proof of concept of edible vaccines. Science 268:714-716.

Holmgren, J., C. Czerkinsky, N. Lycke, and A. M. Svennerholm. 1994. Strategies for the induction of immune responses at mucosal surfaces making use of cholera toxin B subunit as immunogen, carrier, and adjuvant. Am. J. Trop. Med. Hyg. 50:42-54.

Hood, E. E., D. R. Withcher, S. Maddock, T. Meyer, C. B. M. Baszczynski, P. Flynn, J. Register, L. Marshal, D. Bond, E. Kulisek, A. Kusnadi, R. Evangelista, Z. Nikolov, C. Wooge, R. J. Mehigh, R. Hernan, W. K. Kappel, D. Ritland, P. C. Li, and J. A. Howard. 1997. Commercial production of avidin from transgenic maize: characterization of transformant, production, processing, extraction and purification. Molecular Breeding 3:291-306.

Ishida, Y., H. Saito, S. Ohta, Y. Hici, T. Komari, and T. Kumashiro. 1996. High efficiency transformation of maize (Zea mays L.) mediated by Agrobacterium tumefaciens. Nat. Biotechiol. 14:745-750.

Kapusta, J., A. Modelska, M. Figlerowicz, T. Pniewski, M. Letellier, O. Lisowa, V. Yusibov, H. Koprowski, A. Plucienniczak, and A. B. Legocki. 1999. A plant-derived edible vaccine against hepatitis B virus. FASEB J. 13:1796-1799.

Kusnadi, A., R. Evangelista, E. E. Hood, J. A. Howard, and Z. Nikolov. 1998. Processing of transgenic corn seed and its effect on the recovery of recombinant □-glucuronidase. Biotechnol. Bioeng. 60:44-52.

Kusnadi, A. R. 1998. Production and purification of two recombinant proteins from transgenic corn. Biotechnol. Prog. 14:149-155.

Lu, X., H. Kiyono, D. Lu, S. Kawabata, J. Torten, S. Srinivasan, P. J. Dailey, J. R. McGhee, T. Lehner, and C. J. Miller. 1998. Targeted lymph-node immunization with whole inactivated simian immunodeficiency virus (SIV) or envelope and core subunit antigen vaccines does not reliably protect rhesus macaques from vaginal challenge with SIVmac251. AIDS 12:1-10.

Mason, H. S., J. M. Ball, J. J. Shi, X. Jiang, M. K. Estes, and C. J. Arntzen. 1996.

Expression of Norwalk virus capsid protein in transgenic tobacco and potato and its oral immunogenicity in mice. Proc. Natl. Acad. Sci. U. S. A 93:5335-5340.

Mason, H. S., T. A. Haq, J. D. Clements, and C. J. Arntzen. 1998. Edible vaccine protects mice against Escherichia coli heat-labile enterotoxin (LT): potatoes expressing a synthetic LT-B gene. Vaccine 16:1336-1343.

Garvey, P. B., J. Hammond, M. M. Dienelt, D. C. Hooper, Z. F. Fu, B. Dietzschold, H. Koprowvski, and F. H. Michaels. 1995. Expression of the rabies virus glycoprotein in transgenic tomatoes. Biotechnology (N.Y.) 13:1484-1487.

Modelska A, Dietzschold B, Sleysli N, Fu ZF, Steplewski K, Hooper DC, Koprowski H, Yusibov V. 1998 Immunization against rabies with plant-derived antigen. Proc Natl Acad Sci U S A Mar 3;95(5):2481-5.

Moldoveanu, Z., A. N. Vzorov, W. Q. Huang, J. Mestecky, and R. W. Compans. 1999.

Induction of immune responses to SIV antigens by mucosally administered vaccines [In Process Citation]. AIDS Res. Hum. Retroviruses 15:1469-1476.

Pen, J., L. Molendijk, W. J. Quax, P. C. Sijmons, A. J. van Ooyen, P. J. van den Elzen, K. Rietveld, and A. Hoekema. 1992. Production of active Bacillus licheniformis alpha-amylase in tobacco and its application in starch liquefaction.

Biotechnology (N.Y.) 10:292-296.

Ruedl, C. and H. Wolf. 1995. Features of oral immunization. Int. Arch. Allergy Immunol. 108:334-339.

Saif, L. J. and R. D. Wesley. 1992. Transmissible Gastroenteritis, Diseases of Swine 7^{th} Edition, Iowa State University Press 29: 362-386.

Saif, L. J., J. L. van Cott, and T. A. Brim. 1994. Immunity to transmissible gastroenteritis virus and porcine respiratory coronavirus infections in swine. Vet. Immunol. Immumopathol. 43:89-97.

Thanavala, Y., Y.-F. Yang, P. Lyons, H. S. Mason, and C. J. Arntzen. 1995.

Immunogenicity of transgenic plant-derived hepatitis B surface antigen. Proc. Natl. Acad. Sci. U. S. A 92:3358-3361.

Trudel, J., C. Potvin, and A. Asselin. 1992. Expression of active hen egg white lysozyme in transgenic tobacco. Plant Sci. 87:55-67.

Wigdorovitz, A., C. Carrillo, M. J. Dus Santos, K. Trono, A. Peralta, M. C. Gomez, R. D. Rios, P. M. Franzone, A. M. Sadir, J. M. Escribano, and M. V. Borca. 1999. Induction of a protective antibody response to foot and mouth disease virus in mice following oral or parenteral immunization with alfalfa transgenic plants expressing the viral structural protein VP1. Virology 255:347-353.

All references cited herein are hereby expressly incorporated in their entirety by reference.

1. A method of eliciting protection from a pathogen causing disease in an animal comprising: administering to said animal an immunogenic composition at a dosage level so that an antibody response is not observed.

2. The method of claim 1 wherein said pathogen is a viral pathogen.

3. A method of eliciting protection against challenge from a pathogen causing disease in an animal without developing serum level antibodies in said animal comprising:
preparing a vaccine material having edible plant portions of a transgenic maize plant which expresses an antigenic protein of a pathogen;
orally administering an amount of said vaccine material that is less than an amount sufficient to develop serum level antibodies against said pathogen, wherein said protection is characterized by a reduced titer of virus infection upon a subsequent challenge to said pathogen, thereby conferring immunity to said animal compared to an animal not administered said vaccine material.

4. A method of eliciting protection from a pathogen causing disease in an animal comprising: orally administering to said animal an immunologic composition wherein said composition comprises a bacterial enterotoxin capable of eliciting and increased interferon level in said animal and a carrier and wherein said administration is in an effective amount so that an antibody response is not observed.

5. The method of claim 4 wherein said pathogen is a viral pathogen.

6. The method of claim 4 wherein said enterotoxin is a heat labile enterotoxin from Escherichia coli.

7. The method of claim 4 wherein said enterotoxin is LT toxin or its subunits.

8. The method of claim 4 wherein said enterotoxin comprises a mutation to inactivate the A subunit.

9. The method of claim 4 wherein said virus is TGEV.

10. The method of claim 4 wherein said animal is a pig.

11. A method of increasing interferon levels in animals so that a protective effect against pathogens is observed, said method comprising: introducing to said animal in oral form an interferon stimulating amount of an immunologic composition, said composition comprising a bacterial enterotoxin; and a carrier.

12. The method of claim 11 wherein said pathogen is a viral pathogen.

13. A method of increasing interferon levels in animals so that a protective effect against viral pathogens is observed, said method comprising: introducing to said animal in oral form an interferon stimulating amount of an immunologic composition, said composition comprising a bacterial enterotoxin; and a carrier.

13. A method of increasing interferon levels in animals so that a protective effect against viral pathogens is observed, said method comprising: introducing to said animal, in oral form, an interferon stimulating amount of an immunologic composition, said composition comprising a bacterial enterotoxin; and a carrier.

14. The method of claim 13 wherein said enterotoxin is a heat labile enterotoxin from Escherichia coli.

15. The method of claim 13 wherein said enterotoxin is LT toxin.

16. The method of claim 13 wherein said enterotoxin comprises a mutation to inactivate the A subunit.

17. The method of claim 13 wherein said virus is TGEV.

18. The method of claim 13 wherein said animal is a pig.

19. A method of inducing protection in an animal from a disease state caused by a rotavirus and coronovirus infection comprising: orally administering to said animal an alpha interferon stimulating amount of a bacterial enterotoxin.

20. The method of claim 19 wherein said enterotoxin is a heat labile enterotoxin from Escherichia coli.

21. The method of claim 19 wherein said enterotoxin is LT toxin.

22. The method of claim 19 wherein said enterotoxin comprises a mutation to inactivate the A subunit.

23. The method of claim 19 wherein said virus is TGEV.

24. The method of claim 19 wherein said animal is a pig.

25. (Cancelled)

26. A method of eliciting protection from a viral pathogen causing disease in an animal comprising: orally administering to said animal an immunologic composition wherein said composition comprises a bacterial enterotoxin capable of eliciting an increased interferon level in said animal; and a carrier and wherein said administration is in an effective amount so that an antibody response is not observed, wherein said viral pathogen is selected from the group consisting of: TGEV, PRRS, and arterovirus.

27. A method of increasing interferon levels in animals so that a protective effect against viral pathogens is observed, said method comprising: introducing in oral form to said animal an interferon stimulating amount of an immunologic composition comprising a bacterial enterotoxin; and a carrier.

28. A method of inducing protection in an animal from a rotavirus and coronovirus infection comprising: administering to said animal an alpha interferon stimulating amount of a bacterial enterotoxin.

29. The method of claim 3 wherein said pathogen is transmissible gastroenteritis virus (TGEV).

30. The method of claim 3 wherein said animal is a pig.

31. The method of claim 3 further comprising prior to oral administration, determining an amount of vaccine material comprising said plant that will confer immunity without developing serum level antibodies in said animal.

32. The method of claim 30 wherein said pig is administered about 50 grams of said plant per dose.

33. The method of claim 32 wherein said 50 grams of plant comprises about 2 milligram (mg) of a spike protein per dose.

34. The method of claim 29 wherein TGEV has virions expressing spike glyconprotein (S).
